(19) United States
(12) Patent Application Publication
(10) Pub. No.: US 2004/0153095 A1
(43) Pub. Date: Aug. 5, 2004

(54) Method and apparatus for removing urinary tract stones

(76) Inventor: J. Michael Seddon, Dunn, NC (US)

Correspondence Address: COATS & BENNETT, PLLC, P O BOX 5, RALEIGH, NC, 27602, US

(21) Appl. No.: 10/355,590

(22) Filed: Jan. 31, 2003

(51) Int. Cl. A61B 17/22

(52) U.S. Cl. 606/113

(57) ABSTRACT

A method and apparatus for treating kidney stones in the urinary tract uses a surgical assembly to remove kidney stones. The surgical assembly is inserted into the urinary tract along a guide wire positioned in the surgical assembly. Fluid, such as lidocaine gel and contrast solution, and a stone-removing device enter the urinary tract via channels located in the surgical assembly. Once the surgical assembly is proximate the stone, the physician engages and retains the stone with the stone-removing device. An exemplary stone-removing device includes an expandable spacer and an adjustable stone retainer. When in an expanded position, the spacer engages a portion of the urinary tract. The stone retainer, confined within the expanded spacer, engages and retains the stone while the spacer spaces the urinary tract from the stone retainer. The stone is removed by removing the surgical assembly containing the stone from the urinary tract.

### BACKGROUND OF THE INVENTION

The present invention generally relates to a method and apparatus for treating stones in the urinary tract of a patient, and more particularly to a method and apparatus for capturing and retaining a urinary tract stone while removing the stone from the urinary tract.

The urinary tract, comprising the kidneys, ureters, bladder, and urethra, functions to remove waste and extra water from the body. Kidneys, bean-shaped organs approximately the size of a fist, sift waste and extra water from the blood stream. The waste and extra water becomes urine, which flows through the ureters and into the bladder, where it is stored. Once the bladder becomes full, the urine exits the body via the urethra.

Normally, the urine contains chemicals to prevent crystalline build-up within the kidneys. However, sometimes these chemicals are either ineffective or absent from the urine. In these instances, crystalline build-up may occur within the kidneys, forming urinary tract stones. Often such stones exit the body unnoticed. However, if a stone becomes large, it may have difficulty passing through the ureters or the urethra and cause great pain to the individual and/or possible damage to the urinary tract.

There are several techniques available for treating painful urinary tract stones. Surgery, medication, increased fluid intake, Extracorporeal Shock Wave Lithotripsy (ESWL), Electro-Hydraulic Lithotripsy (EHL), and Holmium laser comprise some common treatment methods. While medication effectively treats some urinary tract stones, it is generally ineffective in treating calcium-based stones, the most common type of urinary tract stone in the United States. When medication and/or increased fluid intake are ineffective, ESWL, EHL, and holmium laser become the preferred treatment options in most cases because of the reduced recovery time as compared to surgery. EHL and holmium laser, particularly effective for treating stones located in the ureter, are commonly used to treat larger stones and/or when ESWL fails.

One traditional treatment option uses a ureteroscope to locate the urinary tract stone. A surgeon passes the ureteroscope, a small thin endoscope, through the urethra and bladder into the ureter. After visualizing the stone, the surgeon may capture the stone with a cage-like device passed into the ureter through the ureteroscope. The physician then pulls the cage-like device containing the stone through the ureter, bladder, and urethra, to remove the stone from the body. While effective, this method is not usually used to treat stones located in the upper urinary tract, i.e., the kidneys and upper ureters, because of the danger of injury to the ureters and kidneys during the extraction of the stone. Use of a flexible ureteroscope to monitor the extraction can help minimize damage to the urinary tract. However, introduction of this instrument increases the amount of instrumentation and cost of the procedure. A method whereby the stone can be extracted under x-ray control, aided by lubrication, without the need for a flexible ureteroscope, would be advantageous.

Therefore, there is a need for a method and apparatus that enables a physician to visualize a urinary tract stone, particularly a stone located in the upper urinary tract, while guiding a stone removing apparatus towards the stone and monitoring the removal of the stone and capturing apparatus.

### SUMMARY OF THE INVENTION

The present invention is directed to a method and apparatus for removing stones from the urinary tract of a patient. In an exemplary embodiment, a surgical assembly is used to remove the stone from the urinary tract. The surgical assembly includes a flexible sheath and an introducer positioned within the flexible sheath. The introducer comprises a flexible rod that includes a series of openings, including a guide wire channel to accommodate a guide wire, an irrigation channel for administering fluids to the urinary tract, and an instrument channel for receiving a stone-removing device.

An exemplary stone-removing device according to the present invention comprises an expandable spacer, an adjustable stone retainer, and a controller. The controller expands the spacer from a collapsed position to an expanded position. When in the expanded position, the spacer engages portions of an interior wall of the urinary tract. The controller also adjusts the stone retainer between a collapsed position and an expanded position. Further, the stoner retainer is confined within the spacer when the spacer is in the expanded position.

### BRIEF DESCRIPTION OF THE DRAWINGS

FIG. 1 illustrates a surgical assembly according to the present invention.

FIG. 2 is a cross-sectional view of the surgical assembly taken through the lines 2-2 of FIG. 1.

FIG. 3 illustrates an exemplary stone-removing device.

FIG. 4 illustrates the stone-removing device of FIG. 3 in an expanded configuration.

FIG. 5A illustrates the stone-removing device inserted in a urinary tract and proximate a stone.

FIG. 5B illustrates the stone-removing device in the urinary tract and in an expanded configuration.

FIG. 5C illustrates engaging the stone with the stone-removing device.

FIG. 5D illustrates adjusting the stone-removing device to capture the stone.

FIG. 5E illustrates the stone captured in the stone-removing device.

FIG. 6A illustrates a side cross-section of the surgical assembly of FIG. 1 proximate a stone in the urinary tract.

FIG. 6B illustrates a stone-removing device utilized with the surgical assembly of FIG. 3A.

FIG. 6C illustrates the stone-removing device engaging the stone while the introducer is withdrawn from the surgical assembly.

FIG. 6D illustrates removing the stone with the stone-removing device and the surgical assembly after the introducer has been removed from the surgical assembly.

### DETAILED DESCRIPTION OF THE INVENTION

Urinary tract stones may form anywhere in the urinary tract, including the kidneys and upper sections of the ureters. Some sections of the ureters, such as those sections near the iliac vessels, are particularly narrow, making it difficult to pass stones that form in the upper sections of the urinary tract, i.e., portions of the urinary tract including the kidneys and upper ureters above the iliac vessels. Because traditional basket extraction techniques utilize rigid extraction devices with limited functionality, traditional devices are unable to reach the upper section of the ureters. A flexible ureteroscope can be used in the upper urinary tract to help extract a stone. However, flexible ureteroscopes have a limited use-related life span, particularly when instruments, such as stone basket extraction devices, are repeatedly passed along it. Further, flexible ureteroscope are twice as expensive and have a minimal life span compared to rigid ureteroscopes. Therefore, for the most part, traditional basket extraction techniques are only used to remove stones from the bladder, urethra, and lower sections of the ureters. The disclosure below describes methods and apparatus that overcome the limitations of traditional stone extraction techniques and enable a physician to extract stones from the upper and lower sections of the urinary tract.

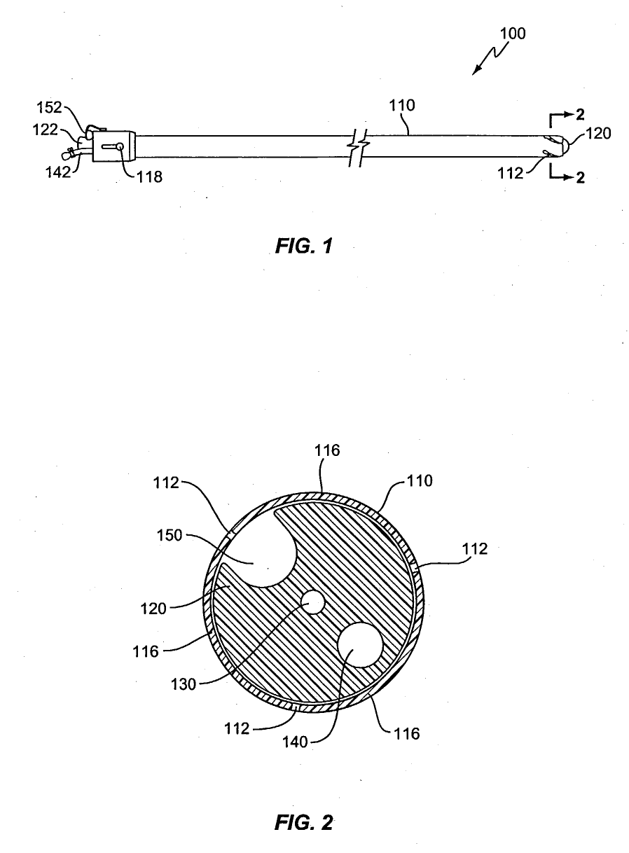

FIG. 1 illustrates a ureteric surgical device 100. Ureteric surgical device 100 comprises a long, hollow, and flexible sheath 110 and an introducer 120 that fits within sheath 110. Introducer 120 comprises a long flexible rod, with a handle 122 at one end, and includes a series of openings such as a guide wire channel 130, an irrigation channel 140, and an instrument channel 150. Sheath 110 includes a tip and a base, with the tip having a series of diagonal slits 112 that enable the tip to expand or flair outwardly. The base includes an external pin 118 for securing introducer 120 to sheath 110 and an instrument channel plug 152 for sealing instrument channel 140 when not in use. While the present invention only describes three introducer openings, those skilled in the art will recognize that introducer 120 may include additional or fewer openings without departing from the scope of the invention.

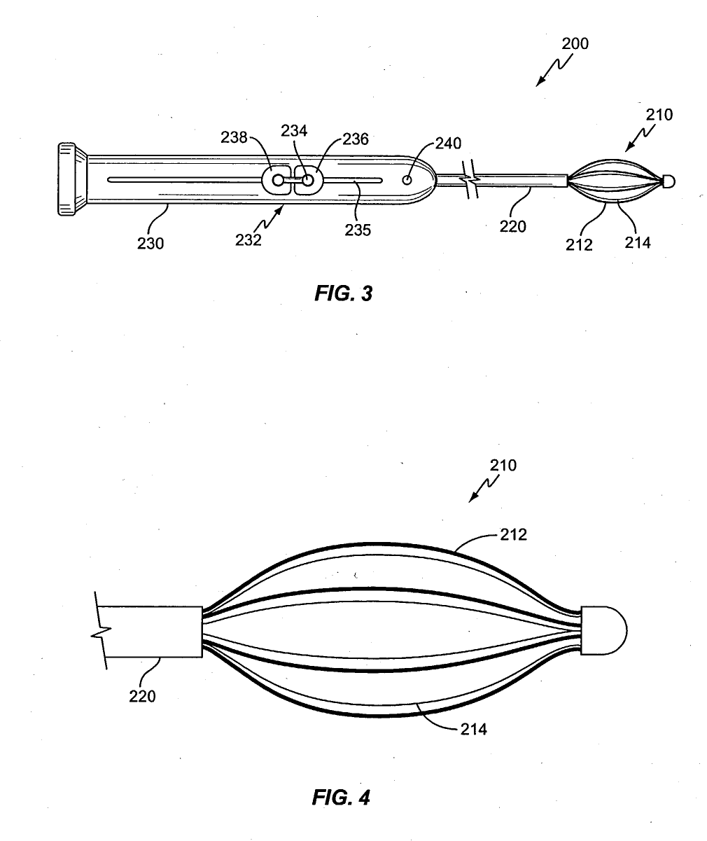

FIG. 2 illustrates a cross-section of the tip of ureteric surgical device 100 and shows the openings in introducer 120. In the embodiment illustrated in FIGS. 1 and 2, sheath 110 includes three equally spaced diagonal slits 112 that form three flanges 116. Slits 112 cut diagonally into the tip of sheath 110 such that each flange 116 provides structural support for adjacent flanges 116. As a result, the tip of sheath 110 tends to expand when an object larger than the diameter of sheath 110 is pulled into sheath 110.

Exemplary embodiments of the introducer 120 position guide wire channel 130 proximate the center of introducer 120, irrigation channel 140 between guide wire channel 130 and the periphery of introducer 120, and instrument channel 150 along the periphery of introducer 120. Instrument channel 150 is partially defined by an elongated groove that runs along the side of introducer 120 and is partially defined by an inner wall of sheath 110. As described in further detail below, this feature enables a physician to remove introducer 120 from the sheath 110 while leaving an instrument within the sheath 110.

Instrument channel 150 is designed to accommodate a wide variety of stone-removing devices. Such devices include EHL probes, laser probes, and conventional stone-removing devices that use helical baskets and Segura baskets. Instrument channel 150 will also accommodate a kidney stone disintegrator of the type described in Applicant's patent application Ser. No. 10/299,432, filed 19 Nov. 2002 and which is herein expressly incorporated by reference.

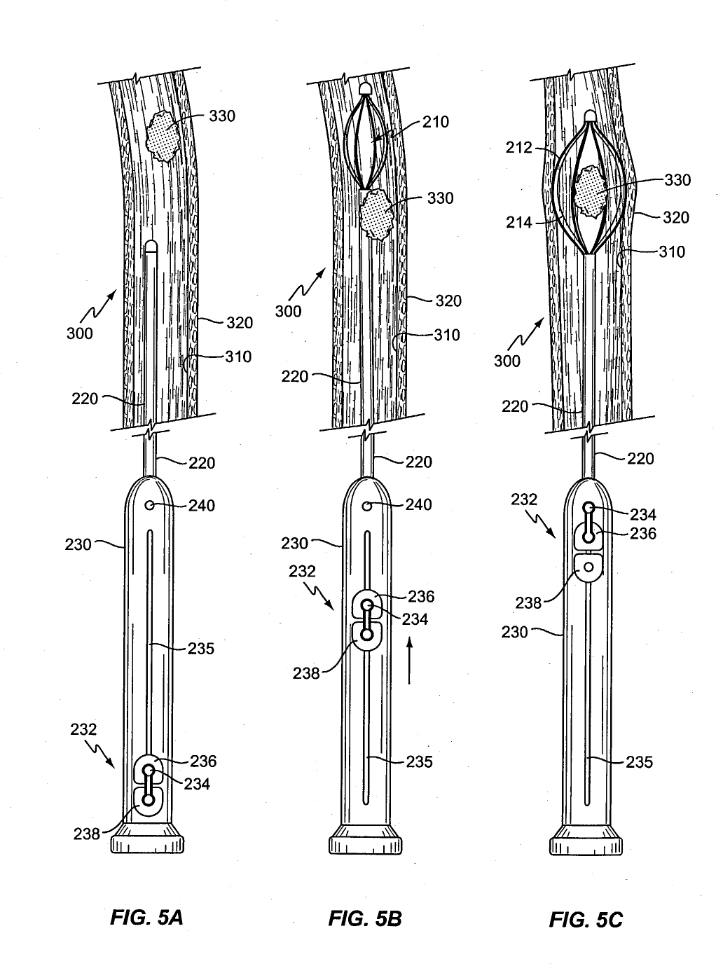

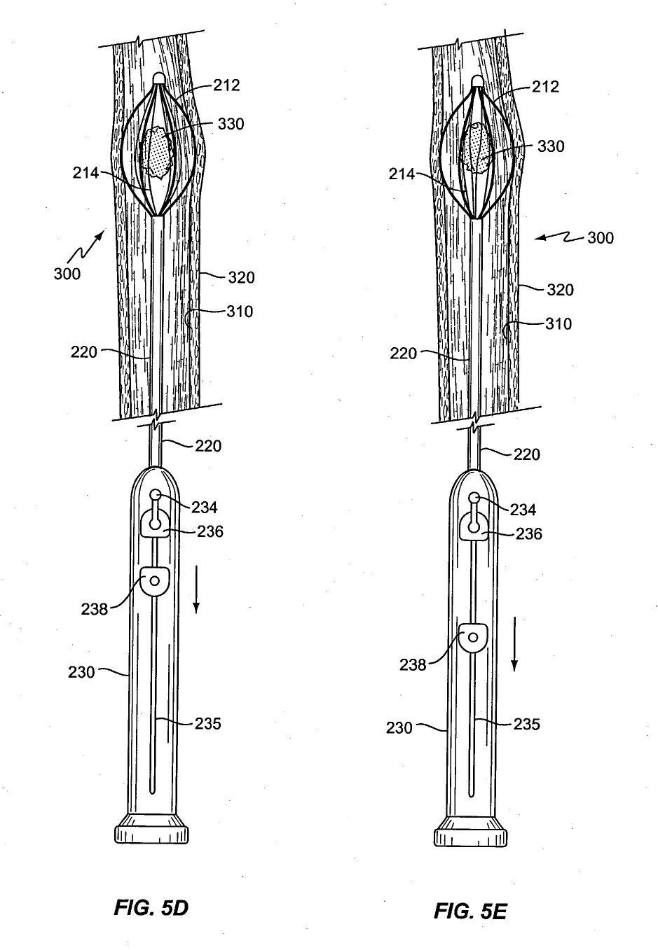

FIGS. 3 and 4 illustrate another exemplary stone-removing device 200 according to the present invention. Stone-removing device 200 includes a basket assembly 210 connected to a handle 230 via an extension sheath 220. Handle 230 includes an upper locking hole 240 and a controller 232 for controlling basket assembly 210. Controller 232 includes a pair of sliding actuators 236 and 238, and a locking or connecting mechanism 234 for selectively connecting the sliding actuators.

Basket assembly 210 consists of a spacer 212 and a stone retainer 214, which are independently controllable. That is, the stone retainer 214 can be moved independently of the spacer 212. For example, and as will be described later, when spacer 212 assumes an expanded position, such as shown in FIG. 4, stone retainer 214 can be moved from an extended position to a collapsed position, while still being confined within spacer 212.

In the case of the embodiment illustrated, both the spacer 212 and the stone retainer 214 are in the form of expandable baskets, such as helical or Segura baskets, that are of an open or mesh construction when in the expanded configuration. Details of the construction of each of the baskets is not dealt with herein because such is not per se material to the present invention and, further, individual basket construction is well known in the art. However, it will be noted that in one embodiment, the material forming the stone retainer 214 may be somewhat more pliable or flexible than the material forming the spacer 212.

Basket assembly 210 is operated by the movement of slide actuators 238, 236 fixed together by pin 234, which, in the case of the embodiment illustrated herein, is a thumb slide that can slide back and forth along an elongated slot 235 formed in handle 230. As actuators 236, 238 are moved from left to right, as viewed in FIG. 3, basket assembly 210, after exiting the sheath 220, expands. Opposite movement, that is, movement from right to left as viewed in FIG. 3, causes basket assembly 210 to contract and move towards a collapsed position.

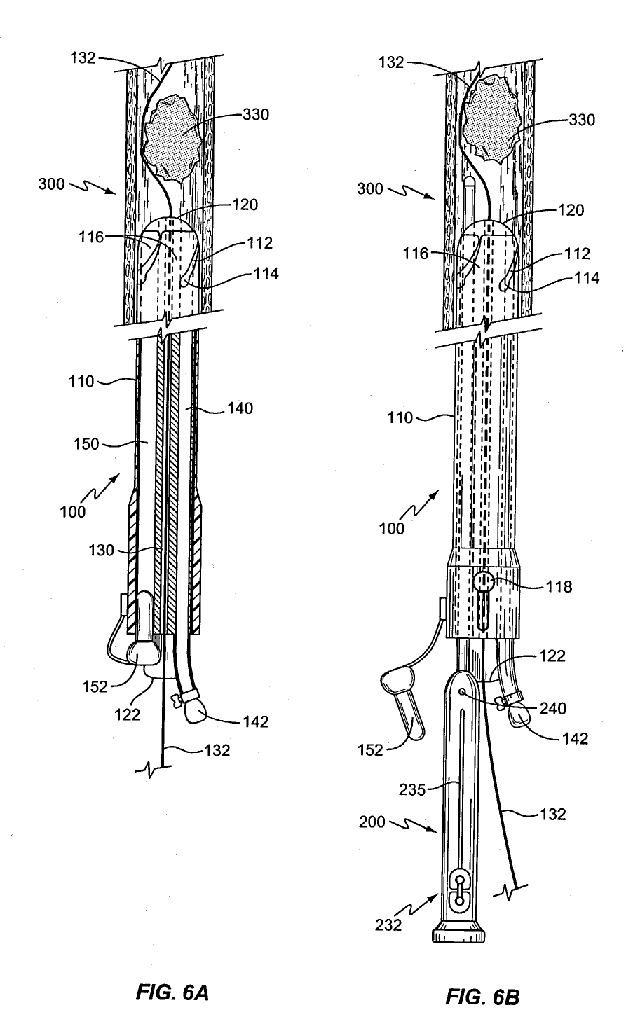

When locking mechanism 234 connects actuators 236 and 238, it follows that the actuators can be moved together back and forth within slot 235. This causes both the stone retainer 214 and the spacer 212 to expand and contract together. However, one feature of the present invention entails the ability to move and control stone retainer 214 independently of spacer 212. For example, by pushing both actuators 236 and 238 to the upper portion of slot 235, as viewed in FIGS. 5A-5E, it follows that both the stone retainer 214 and the spacer 212 are fully expanded. Spacer 212 can then be locked in this expanded position by securing the spacer actuator 236 to the upper locking hole 240 with locking mechanism 234. Now, by moving actuator 238, the stone retainer 214 can be adjusted, independently of the spacer 212, within the confines of the spacer 212. 212 in the expanded position. At this time, a surgeon utilizing the stone removing device 200 is ready to manipulate basket assembly 210 around the stone. In doing so, the basket assembly 210 is positioned against the stone 330 such that stone 330 is urged through spacer 212 and into the confines of the stone retainer 214. This is illustrated in FIG. 5C. After the stone 330 is within the confines of stone retainer 214, then actuator 238, as viewed in FIG. 5D, is moved downwardly along slot 235. This controls the stone retainer 214 independently of spacer 212. That is, the downward movement of actuator 238 causes stone retainer 214 to contract around stone 330. Stones 330 frequently have disparate axes and naturally, the narrowest axis aligns itself with the lumen of the ureter. As the stone retainer 214 contracts around stone 330, the opening within stone retainer 214 tends to become smaller and, consequently, stone 330 is securely held and cannot move outside of the confines of the stone retainer 214. As a result, stone retainer 214 ensures the narrowest axis of stone 330 maintains its alignment during extraction. Note that once spacer 212 assumes an expanded position it generally engages the inner wall 310 of the urinary tract 300. This effectively disengages inner wall 310 from stone 330. With spacer 212 in its expanded position, and with stone 330 securely captured in stone retainer 214, the physician can then remove basket assembly 210, including stone 330 spaced from the inner wall 310 by spacer 212, from the urinary tract 300.

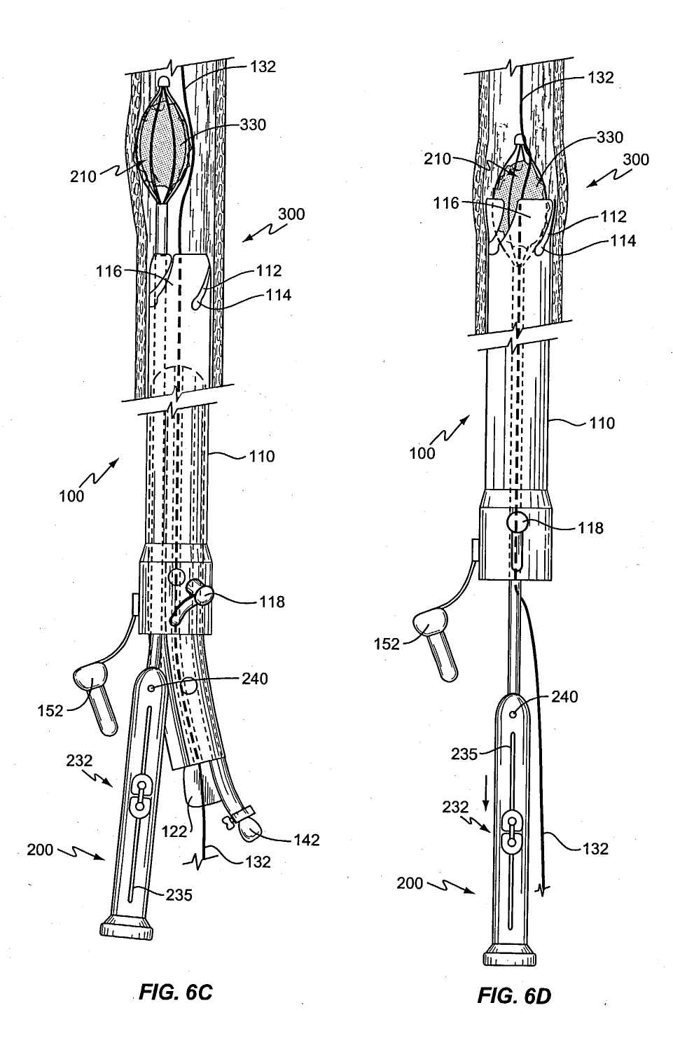

FIGS. 6A-6B illustrate an exemplary method of using surgical device 100 to remove stones 330 from the urinary tract 300 of a patient. After inserting a guide wire 132 into urinary tract 300, a physician positions guide wire 132 within guide wire channel 130 and inserts the surgical device 100 into the urinary tract 300 along guide wire 132 (FIG. 6A). While inserting surgical device 100, introducer locking pin 118 secures introducer 120 to sheath 110. Fluid injected into irrigation port 142 enters urinary tract 300 via the irrigation channel 140. The fluid may include a saline solution, such as lidocaine jelly mixed with saline to treat any ureteric spasms. In addition, the fluid may comprise a contrast solution. Contrast solution injected into urinary tract 300 enables a physician to visualize urinary tract 300 and ureteric stone 330 with an x-ray, or some other external viewing device, while inserting surgical device 100 into urinary tract 300.

Once positioned immediately distal to stone 330, instrument channel plug 152 is removed and stone-removing device 200 is inserted into the instrument channel 150 to position the stone-removing device 200 proximate stone 330 (FIG. 6B). While FIG. 6B illustrates inserting the stone-removing device 200 of FIGS. 3 and 4 into instrument channel 150, those skilled in the art will recognize that other stone removing instruments may also be used.

After engaging and capturing stone 330 with basket assembly 210, the physician removes introducer-locking pin 118 and withdraws the introducer 120 from sheath 110 using handle 122 (FIG. 6C). Because the inner wall of sheath 110 bounds part of instrument channel 150, the physician may also completely remove introducer 120 from sheath 110, without disturbing the relative positions of guide wire 132 and stone-removing device 200, by bending the introducer 120 away from handle 230 of stone-removing device 200 (FIGS. 6C & 6D). At this point in time, surgical device 100 provides the surgeon with three main options:

1. Complete removal of the introducer 120 from sheath 110 followed by extraction of the stone 330 through sheath 110 with stone-removing device 200. The stone 330 should enter the sheath 110 without causing the expansion of the tip of sheath 110.

2. Complete removal of the introducer 120 from sheath 110 followed by introduction of a flexible ureteroscope (not shown) through sheath 110. The stone 330 is then removed from the urinary tract 300 under endoscopic control.

3. Partial retraction of introducer 120 into sheath 110 followed by retraction of stone 330 contained in stone-removing device 200 into the tip of sheath 110. When stone 330 is larger than the tip of sheath 110, the tip flares outwards, due to slits 112, to accommodate stone 330 into the tip of sheath 110 (FIG. 6D). When introducer 120 is only partially withdrawn into sheath 110 and stone 330 is withdrawn into the tip of sheath 110 (not shown), fluids injected into irrigation port 142 are administered to the urinary tract 300 via irrigation openings 114 located at the base of slits 112. The fluid should contain contrasting material. It may also contain medications to lubricate and/or control ureteric spasms. See FIG. 6D. Once stone 330 is positioned in the tip of sheath 110, the physician removes surgical device 100, including stone-removing device 200 and the captured stone 330, from the urinary tract 300 of the patient under x-ray control.

The above described apparatus and methods provide several advantages over traditional treatment options. First, flexible sheath 110 and introducer 120 of ureteric surgical device 100 enable a physician to position the ureteric surgical device 100 anywhere within urinary tract 300, including the upper sections of the ureters and the kidneys. As a result, a physician has access to kidney stones located anywhere within urinary tract 300. Further, the multiple channels 130, 140, 150 of introducer 120 enable a physician to visualize, capture, and remove stones 330 without using an endoscope and without repeatedly removing and inserting one or more devices. In addition, introducer 120 may be removed from the urinary tract 300 without removing any stone-removing devices 200. This feature, along with the slits 112 in the tip of sheath 110 enables the stone 330 to be at least partially withdrawn into sheath 110, making it easier to extract stone 330 from the urinary tract 300. Lastly, spacer 212 of stone-removing device 200 spaces kidney stone 330 from any surrounding tissue of the urinary tract 300, also making it easier to extract stone 330. The features of the above described methods and apparatus, alone or in combination, ultimately provide a more efficient stone-removing technique and device.

Although the apparatus of the present invention has been described in connection with removing urinary tract stones, it will be appreciated by those skilled in the art that the present invention can be utilized in other areas of the bodies of humans and animals to remove problematic particles and objects.

The foregoing description and drawings describe and illustrate the present invention in detail. However, the foregoing only describes some embodiments of a urinary tract stone remover. Accordingly, the present invention may be carried out in specific ways other than those set forth herein without departing from the essential characteristics of the invention. The present embodiments are, therefore, to be considered in all respects as illustrative and not restrictive, and all changes coming within the meaning and equivalency range of the appended claims are intended to be embraced therein.

What is claimed is:

1. A surgical assembly for removing a stone from a urinary tract comprising:
a flexible sheath comprising an elongated internal opening; and
an introducer positioned within the elongated internal opening of the flexible sheath comprising:
a guide wire channel to accommodate a guide wire;
an irrigation channel for administering fluids to the urinary tract; and
an instrument channel for receiving a device to remove the stone from the urinary tract.

2. The surgical assembly of claim 1 wherein the instrument channel comprises a groove along a periphery of the introducer.

3. The surgical assembly of claim 2 wherein the groove is open to an inner wall of the sheath such that the groove and the inner wall of the sheath define the instrument channel.

4. The surgical assembly of claim 1 wherein the sheath further comprises a first end and a second end and wherein the first end comprises a plurality of slits spaced around said first end.

5. The surgical assembly of claim 4 wherein the plurality of slits comprise a plurality of diagonal slits.

6. The surgical assembly of claim 4 wherein at least one of the plurality of slits includes an irrigation opening for administering fluids to the urinary tract.

7. The surgical assembly of claim 1 wherein the introducer further comprises a handle for adjusting the position of the introducer within the sheath.

8. The surgical assembly of claim 1 further comprising a locking mechanism for selectively securing the introducer to the sheath.

9. The surgical assembly of claim 1 wherein the guide wire channel is centrally located within the introducer.

10. The surgical assembly of claim 1 wherein the device received by the instrument channel comprises a stone-removing device.

11. The surgical assembly of claim 10 wherein the stone-removing device comprises at least one of a stone disintegrator and a stone retainer.

12. A method for engaging and retaining a stone from a urinary tract of a patient with a surgical assembly, the surgical assembly including a flexible sheath comprising an elongated internal opening and an introducer positioned within the elongated internal opening of the sheath, wherein the introducer includes a guide wire channel, an irrigation channel, and an instrument channel, the method comprising:
guiding the surgical assembly through the urinary tract of the patient to an area proximate the stone along a guide wire positioned in the guide wire channel;
administering fluid into the urinary tract of the patient via the irrigation channel;
inserting a stone removing device into the urinary tract of the patient via the instrument channel; and
engaging and retaining the stone with the stone removing device.

13. The method of claim 12 wherein the urinary tract further comprises:
a lower section, including the urethra, bladder, and a section of the ureter below the iliac vessels;
an upper section, including the kidneys and the portion of the ureter above the iliac vessels; and
wherein guiding the surgical assembly comprises guiding the surgical assembly through the lower section and into the upper section.

14. The method of claim 12 further comprising retracting the introducer within the sheath while maintaining the relative positions of the stone removing device and the guide wire.

15. The method of claim 14 wherein the sheath further comprises a tip comprising a plurality of slits spaced around the tip, the method further comprising:
retracting at least a portion of the stone retained in the stone removing device into the tip of the sheath, wherein the plurality of slits allow the tip of the sheath to expand as necessary; and
removing the surgical assembly and the stone from the urinary tract.

16. The method of claim 15 wherein at least one of the plurality of slits includes an irrigation opening, the method further comprising administering fluid into the urinary tract via the irrigation opening.

17. The method of claim 14 wherein the sheath further comprises a tip comprising a plurality of slits spaced around the tip, the method further comprising:
disintegrating the stone into a plurality of stone fragments; and
retaining one or more stone fragments with the stone removing device.

18. The method of claim 17 wherein at least one of the plurality of slits includes an irrigation opening, the method further comprising administering fluid into the urinary tract via the irrigation openings.

19. The method of claim 14 further comprising removing the introducer from the sheath while maintaining the relative positions of the stone removing device and the guide wire.

20. The method of claim 19 wherein removing the introducer comprises bending the introducer away from the stone removing device while withdrawing the introducer from the sheath.

21. The method of claim 19 further comprising inserting a second surgical instrument into the sheath after removing the introducer.

22. The method of claim 12 wherein administering fluid comprises administering at least one of a contrast solution and a saline solution into the urinary tract of the patient.

23. An instrument for engaging and retaining a stone within a urinary tract of a patient comprising:
a spacer expandable from a collapsed position to an expanded position and operative to engage an interior wall of the urinary tract when in an expanded position;
a stone retainer adjustable between a collapsed position and an expanded position and confined within the spacer when in the expanded position; and
a controller for moving the spacer and the stone retainer between the collapsed and expanded positions.

24. The instrument of claim 23 wherein the controller comprises a first controller connected to the spacer and a second controller connected to the stone retainer.

25. The instrument of claim 24 wherein each of the first and second controllers are moveable, with each controller being movable with respect to one another such that the stone retainer may be adjusted with respect to the spacer.

26. The instrument of claim 25 further comprising a locking mechanism to selectively lock the first and second controllers together when expanding the spacer and the stone retainer from the collapsed positions to the expanded positions and to selectively secure the spacer in the expanded position.

27. The instrument of claim 26 wherein the second controller adjusts the stone retainer independently of the first controller.

28. The instrument of claim 23 wherein the spacer comprises a basket made of a first material and wherein the stone retainer comprises a basket made of a second material.

29. The instrument of claim 28 wherein the second material is more flexible than the first material.

30. The instrument of claim 28 wherein the spacer basket and the stone retainer basket each comprise a helical basket.

31. The instrument of claim 28 wherein the spacer basket and the stone retainer basket each comprise a Segura basket.

32. A method for removing a stone from a urinary tract of a patient comprising:
inserting a collapsed spacer and a collapsed stone retainer into the urinary tract;
expanding the spacer and engaging a wall of the urinary tract with the spacer;
expanding the stone retainer within the confines of the expanded spacer; and
capturing one or more stones with the stone retainer.

33. The method of claim 32 wherein capturing the one or more stones with the stone retainer comprises independently adjusting the stone retainer with respect to the spacer to retain the stone within the stone retainer.

34. The method of claim 32 further comprising removing the expanded spacer and the stone captured by the stone retainer from the urinary tract.
